(19) United States
(12) Patent Application Publication
(10) Pub. No.: US 2006/0015030 A1
(43) Pub. Date: Jan. 19, 2006

(54) Method for placing multiple implants during a surgery using a computer aided surgery system

(75) Inventors: Francois Poulin, Montreal (CA); Louis-Philippe Amiot, Montreal (CA)

Correspondence Address: OGILVY RENAULT LLP, 1981 MCGILL COLLEGE AVENUE, SUITE 1600, MONTREAL, QC, H3A2Y3, CA

(73) Assignee: ORTHOSOFT INC., Montreal (CA)

(21) Appl. No.: 10/526,018

(22) PCT Filed: Aug. 25, 2003
(86) PCT No.: PCT/CA03/01248

§ 371 Date: Feb. 25, 2005

Related U.S. Application Data
(60) Provisional application No. 60/405,703, filed on Aug. 26, 2002.

(51) Int. Cl. A61B 5/05

(52) U.S. Cl. 600/424

(57) ABSTRACT

A method and an apparatus are described for placing multiple implants during a surgery, the apparatus comprising: a display for an image representing a patient's anatomy; a database of virtual implants from which a user selects; a tool for the user to manipulate in order to select the virtual implants from the database and place the virtual implants in the image at desired locations; and a positioning module for calculating a position of a first of the virtual implants with respect to a second of the virtual implants and allow the user to align the first and second virtual implants with respect to each other, for generating relative position data as a function of the calculated position, and for sending the relative position data to the display.

### FIELD OF THE INVENTION

The invention relates to placing multiple implants during a surgery. More specifically, it relates to aligning multiple virtual implants together using a computer-aided surgery system in order to more precisely place the actual implants.

### BACKGROUND OF THE INVENTION

There are a variety of computer-aided surgery systems that exist for assisting a surgeon in a surgery. Such systems allow the surgeon to view the anatomy of a patient before surgery in order to plan the procedure and during surgery in order to be guided throughout the procedure.

Surgical navigation is based on displaying, in real-time, instruments and patient anatomy to allow unobstructed visualization of the complete surgical field. Patient anatomy can be obtained from a number of sources, such as CT-scan, digitization, fluoroscopy, etc. Patient bone position and orientation are measured in real-time. They are used as references so that patient movement will not impact the navigation accuracy. Instrument position and orientation are also measured in real-time. This is used to display the instrument position relative to the patient bone on the computer screen.

Currently, systems allow surgeons to place a virtual implant in an image, select the implant from a group of virtual implants, and simulate the movement of a bone with the virtual implant. The virtual implant is placed with respect to its target bone. However, when there is more than one implant to be placed, and these implants are related to each other, it would be ideal to align the implants together in an optimal way.

Moreover, since it is essential to place implants with extreme precision, there is a need to provide additional tools to surgeons in order to allow them to optimize the placements of the implants.

### SUMMARY OF THE INVENTION

Accordingly, an object of the present invention is to provide additional planning tools for surgeons within a computer-aided surgery system.

Another object of the present invention is to align multiple virtual implants with respect to each other in an image.

According to a first broad aspect of the invention, there is provided an apparatus for planning a surgery, the apparatus comprising: a display for an image representing a patient's anatomy; a database of virtual implants from which a user selects; a tool for the user to manipulate in order to select the virtual implants from the database and place the virtual implants in the image at desired locations; and a positioning module for calculating a position of a first of the virtual implants with respect to a second of the virtual implants and allow the user to align the first and second virtual implants with respect to each other, for generating relative position data as a function of the calculated position, and for sending the relative position data to the display.

Preferably, calculating a position comprises determining how well the virtual implants fit along a curve representing an interconnecting member for the virtual implants. In a preferred embodiment, the surgery is a spinal surgery, the virtual implants are at least two spinal implants, and the positioning module is for aligning the at least two spinal implants along a curve representing an interconnecting member for the spinal implants.

According to a second broad aspect of the present invention, there is provided a method for placing at least two spinal implants during a surgery using a computer assisted surgery system, the method comprising: providing an image representing a patient's anatomy; determining a desired curve along which the at least two spinal implants are to be placed and representing the curve on the image, the desired curve corresponding to an interconnecting member for the at least two spinal implants; selecting at least two virtual implants from a database of virtual implants to correspond to the at least two spinal implants; placing the at least two virtual implants on the desired curve in the image by aligning the at least two virtual implants with the desired curve while taking into account a position of a preceding virtual implant to place a subsequent virtual implant; and placing the at least two spinal implants according to the virtual implants in the image using the computer assisted surgery system.

Preferably, determining one of a position and a shape of the subsequent virtual implant further comprises using lines to join together the virtual implants and align them on the image representing a patient's anatomy. Alternatively, determining one of a position and a shape of the subsequent virtual implant further comprises calculating a location for the subsequent virtual implant based on a location of the preceding virtual implant. Determining one of a position and a shape of the subsequent virtual implant further comprises constraining the one of a position and a shape based on constraints imposed by the preceding virtual implant.

The method also comprises re-adjusting a position of said preceding virtual implant to better position said subsequent virtual implant in order to achieve an optimal alignment of all of said virtual implants.

Also preferably, the planning module is used with a computer aided surgery system and a tracking module.

According to a third broad aspect of the invention, there is provided a computer data signal embodied in a carrier wave comprising data resulting from a positioning module for calculating a position of a first virtual implant with respect to a second virtual implant and allow a user to align the first and second virtual implants with respect to each other, for generating relative position data as a function of the calculated position, and for sending the relative position data to a display.

There is also provided a computer readable memory for storing programmable instructions for use in the execution in a computer of the method in accordance with the invention.

### BRIEF DESCRIPTION OF THE DRAWINGS

These and other features, aspects and advantages of the present invention will become better understood with regard to the following description and accompanying drawings wherein:

FIG. 1 is an interface image showing three virtual screws in the pedicles;

FIG. 2 is an interface image showing a drill guided by a bull's eye;

FIG. 3 is an interface image showing a straight line used to align two virtual screws;

FIG. 4 is a block diagram of the apparatus;

FIG. 5 is a flow-chart according to the method of the present invention; and

FIG. 6 is a block diagram of a system using the apparatus.

### DETAILED DESCRIPTION OF THE PREFERRED EMBODIMENT

Throughout this application, the preferred embodiment of the present invention will be referred to as a “FluoroSpine™ application” of a “Navitrack™ system”. While the present invention described in more detail below is exemplified by a fluoroscopic image-based system, it is not limited to the described embodiment and could be practiced with many different types of navigation and/or imagery systems.

The Navitrack™ product is a device used intra-operatively to provide the surgeon with additional precise information concerning his maneuvers. In an imagery based application, the product displays patient anatomy (obtained from pre or intra-operative images) and overlays the real time position of the surgeon's instruments. In addition, quantitative data relevant to the surgery is displayed on the screen. The FluoroSpine™ has the objective of giving navigation capabilities for the surgeon's instruments on intra-operative images. This is particularly useful for simple cases that can be operated without the optimal accuracy gained from a more radiation intensive scan like CT (computed tomography) or in trauma situations.

In a preferred embodiment, tracking is done with a POLARIS optical tracking system. POLARIS detects infrared light emitted by an active tracker or reflected by a passive tracker. Three dimensional position in space can only be evaluated when a minimum of three spheres or LEDs are seen by the camera.

The principles of navigation and fluoroscopy are based on a tracked device placed over the intensifier of a C-arm. Plates with lead beads fitted to this device allow to position markers in known locations in the image.

Calibration of the image is done by computing the cone of projected x-rays. This step allows all virtual objects to be projected accurately on the C-arm images. From the markers, the x-ray volume is computed. Depending on the instrument position in the x-ray volume, its appearance may vary.

More specifically, calibration with the fluoroscope is done in several steps. A first shot is taken of an image comprising a plurality of artifacts of known relative positions. The computer detects that an image was taken. The computer asks the tracking system the position and orientation of the clamp and the tracker on the C-arm. The computer then acquires the image. Image processing is done to find the positions of the artifacts with respect to the known position of the C-arm tracker. The system can then extrapolate the position of a cone with respect to the camera reference coordinate system. The position of the cone with respect to the clamp can then be redefined.

An x-ray sensitive diode may be integrated into the system in order to increase the speed of image detection by the system. The goal is to minimize the accuracy reduction caused by patient motion (ex: breathing) when the tracking system records the reference tracker position.

The process for navigated fluoroscopy for spinal operations goes as follows. Patient data is first entered into the system. An awl or drill guide is calibrated, as well as a screw-driver. When the patient has been prepared, a vertebral clamp is placed. Fluoroscopy shots are taken and automatically transferred to the Navitrack™ system. Image calibration is performed automatically by the Navitrack™ system. The calibration of the shots to be used for navigation is then validated. For each necessary screw, the surgeon navigates his tool to position a virtual implant which is used to determine true implant size. The surgeon then leaves this virtual implant in the form of an axis on the navigated images. With the screw-driver, the surgeon navigates the real implant to match the planned axis. The outline of this implant is left on the images. When all screws are placed for the calibrated fluoroscopy shots, the surgeon takes a snapshot of the desired views for intra-operative documentation. Then it is back to the acquisition of fluoroscopy shots to operate the next vertebral segment.

The basic technical steps for this type of application are the following:
Calibration of the surgeon's instruments to the tracking system coordinate system
Image acquisition from the fluoroscope by the navigation system
Dewarping of acquired image
Calibration of image to tracking system coordinate system
Removal of calibration object patterns from images
Navigation of the surgeon's instruments on the fluoroscope images

Calibration of the surgeon's instruments to the tracking system coordinate system: Trackers are included on all the tools that the surgeon will use during the navigation. In order to properly display the information relative to these instruments, it is necessary to establish the mathematical relationship between each tracker and its corresponding tool tip position and orientation. This procedure is called calibration of the instruments. Basically, the tracker's position and orientation is measured by the tracking system while, simultaneously, the tool tip's position and orientation is in a position and orientation known by the tracking system.

Image acquisition from the fluoroscope by the navigation system: Before proceeding with the image acquisition, a calibration object must be installed on the fluoroscope. This frame contains active trackers and 2 radio-transparent plates with a number of radio-opaque beads and/or wires. A calibration procedure is designed to establish the bead/wire position relative to the active tracker.

The Navitrack™ monitors the fluoroscope to detect when a shot is taken. At this moment, the position and orientation of all the trackers must be measured with the tracking system. In any case, the patient reference and calibration object position and orientation must be measured by the tracking system at the time of the shot to allow dewarping and calibration. The image is transferred to the Navitrack™ system via means such as a video cable that connects to the fluoroscope video output.

Dewarping of acquired image: As described in many scientific papers, the fluoroscope images may contain distortions caused by optical characteristics of the system, external magnetic fields, etc. These distortions would reduce the accuracy of the navigation, particularly in the image extremities. It is therefore important to remove these distortions.

The distortion-removing algorithms use some of the beads/wires from the calibration object. Since these beads/wires are contained in the image and placed in a particular pattern, it is possible to determine a mathematical transformation that dewarps the pattern in the image. This transformation is then applied to the remainder of the image. Naturally, to use this algorithm, it is necessary to detect the calibration object beads/wires within the image. To minimize operating room time required from the surgeon, this process is automated.

Calibration of image to tracking system coordinate system: The principle for this calibration is to establish the mathematical relationship between the patterns identified in the image (see previous step) and the beads/wires true position in space. Since the calibration object is tracked and the bead/wire pattern position and orientation relative to the tracker is known (see image acquisition), the beads/wires true position in space is also known.

Removal of calibration object patterns from image: Once the image is dewarped and calibrated, the calibration object patterns are no longer useful to the surgeon and can be removed to insure that the surgeon's view is not limited.

Navigation of the surgeon's instruments on the fluoroscope images: The surgeon will do the steps described above as many times as needed until he has the images required for the surgery. At this point, other objects tracked by the system may be superimposed on the images obtained from the fluoroscope. While this document describes the navigation as intended for a pedicle screw insertion in the lumbar spine, a number of other navigation tools could be designed.

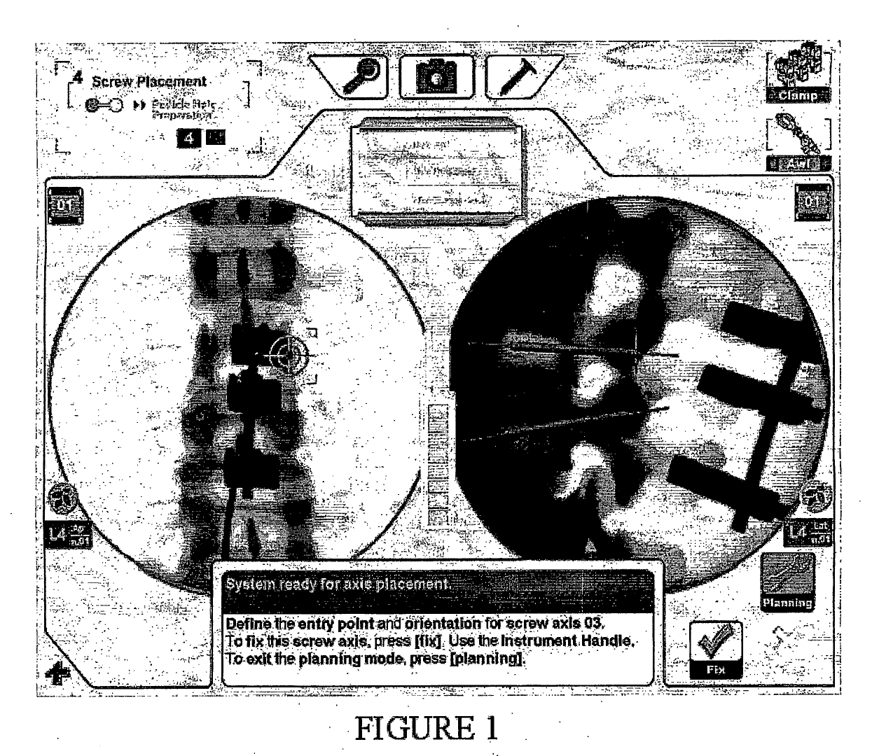

To illustrate the principle of the present invention, the following example is used. A virtual screw is inserted on the computer display of one of the surgeon's tracked tool. This visual representation will be used to plan how the surgeon will position his next screw and which screw size can be used safely. FIG. 1 shows a graphic user interface with three virtual screws in the image. Once the surgeon is satisfied with the virtual screw position, an insertion axis will be displayed on the fluoroscope images to guide the surgeon for the drilling of the holes and the placement of the real screw. This procedure should be followed for all the screws to be placed on vertebrae where the patient reference is placed. When all the screw locations seen in the images have been determined and the screws placed, the surgeon can return to step 2 and repeat all of the following steps until the screws are all inserted. It is possible to save the fluoroscope images with an overlay of the final screw positions in a standard graphic format.

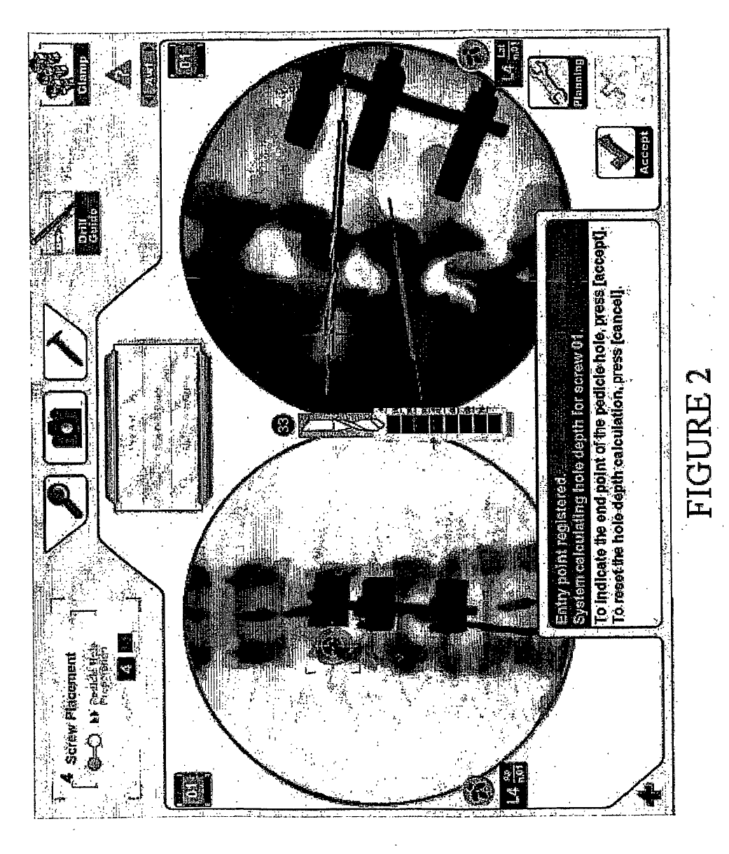

The surgeon may place virtual implants on multiple bones. For example, the surgeon may place virtual screws on all of the vertebrae in one fluoroscopy image. He places his pointing tool on the chosen entrance point for each screw based on his knowledge of the patient's anatomy and his navigation system. On the screen of the navigation system, he can see the virtual screws and adjust the diameter of each screw in order to ensure that the screw will not be larger than the pedicle. The virtual screws can be aligned with respect to the bones, or with respect to each other. The virtual screws can then be fixed in place in the image and graphical tools such as targets or bull's eyes can help the surgeon place the real implant in the planned area. FIG. 2 shows an interface image wherein a bull's eye guides the drill of a surgeon for the placement of the screws.

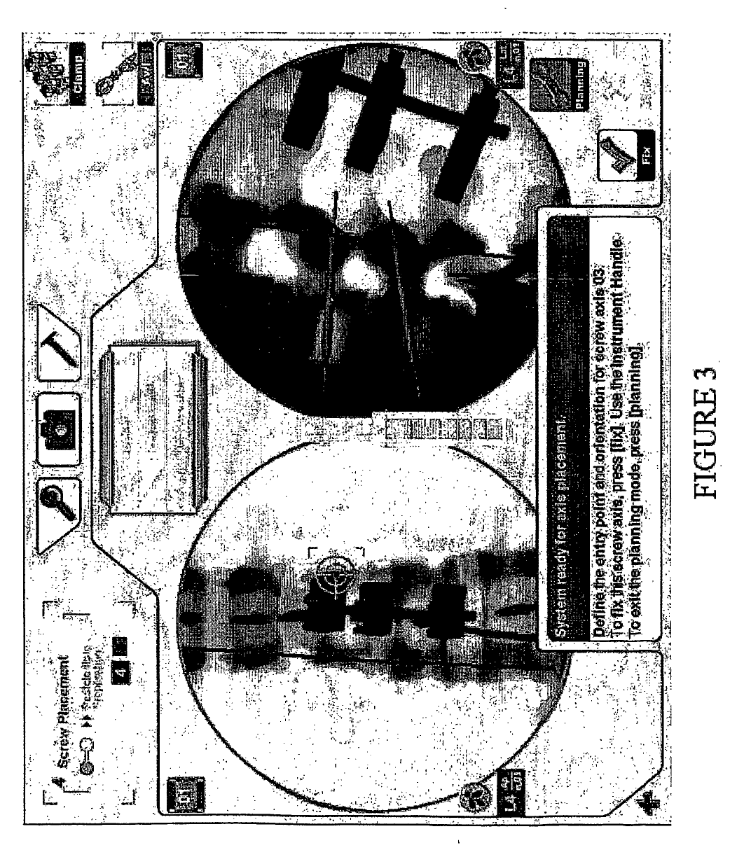

By placing multiple virtual implants in one image, planning tools allow the surgeon to better align the implants. For example, if the goal is to place the virtual screws in a straight line, a line can be traced on the screen between two virtual screws, allowing the surgeon to properly align the subsequent screws and obtain the targeted rectilinear alignment. This can be seen in FIG. 3, where an interface image shows a straight line used to align two virtual screws together. Another example is in the case of multi-implant constructs, such as for scoliosis, which is an abnormal lateral curve of the spine. The surgeon can provide a rod of predetermined shape and the navigation system can then illustrate this rod with respect to the screws in order to indicate the optimal alignment. Alternatively, once the screws are placed, the navigation system can provide the optimal curve for the rod in order to facilitate insertion.

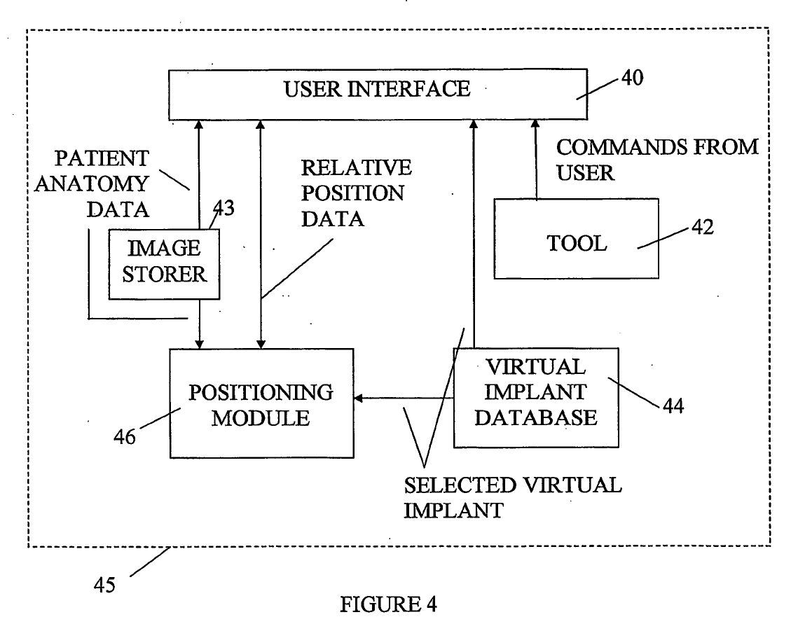

FIG. 4 shows an embodiment of the apparatus according to the invention. A display user interface 40 receives command data from the user via a tool 42 manipulated by the user. The tool 42 can be a pointer which touches the screen directly, a computer mouse that controls a cursor on a display, or any other type of tool that allows the user to interface with the graphics on the display. From the user interface 40, the user can access a database of virtual implants 44. The database 44 comprises all the possible sizes and shapes of implants available for the surgery.

The apparatus also comprises a positioning module 46. The positioning module 46 can detect where a virtual implant has been placed by the user and determine its position in a reference frame. It can also calculate where a second virtual implant should be placed with respect to the position and orientation of the first virtual implant. If two virtual implants have been placed, it can determine where a third virtual implant should be placed in order to match an alignment of the first two virtual implants. If the placement of a third virtual implant is impossible given the anatomy of the patient and the position of the first two virtual implants, the positioning module 46 can group together the first two implants and move them in position and orientation together in order to align them with a placement of the third virtual implant. The positioning module 46 can also calculate what size or shape the third virtual implant should be in order to properly fit with the alignment imposed by the placement of the first two virtual implants. The positioning module 46 can also adjust individually the first two virtual implants in order to better co-exist with the third virtual implant. It can be appreciated that three virtual implants are used to demonstrate the capabilities of the positioning module 46 and should not in any way limit the scope of the module. Relative position data is exchanged between the user interface 40 and the positioning module 46. An image storer 43 comprises images of the patient anatomy and transmits patient anatomy data to the user interface 40 for the user to view and to the positioning module 46 for the module to use the data in its calculations and placement operations. The positioning module 46 can select an ideal virtual implant from the virtual implant database 44.

The tool 42 allows the user to group together two or more virtual implants and input a desired relative position of the group of virtual implants with respect to another virtual implant or another group of implants. The positioning module 46 can then update the position of either the group of virtual implants or the other virtual implant as a function of the desired relative position. The positioning module 46 can also update a position of a first virtual implant after a second virtual implant has been placed as a function of a predetermined relative position criteria. The position module 46 can send relative position data that is graphically or numerically represented on the user interface 40. The relative position data can comprise information related to the entry point of the virtual implant on the anatomy, the orientation of the virtual implant on the anatomy, and depth information of the virtual implant in the anatomy.

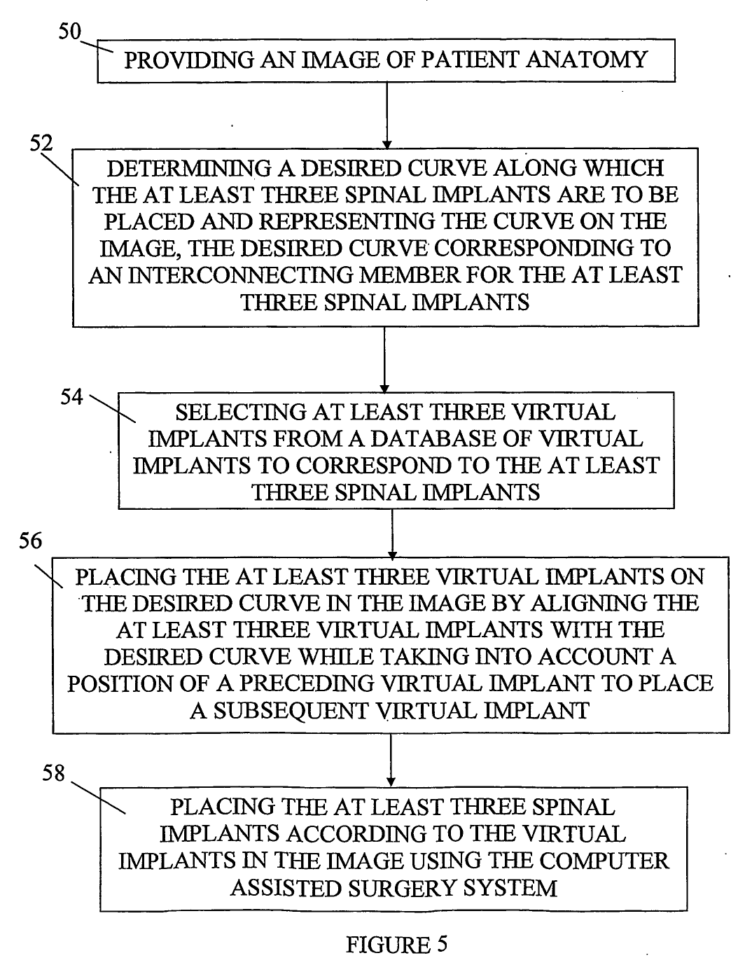

FIG. 5 is a flowchart of the method of the present invention. The first step consists in providing an image of patient anatomy 50. This can be done pre-operatively or intra-operatively. The next step is to determine a desired curve along which the at least three spinal implants are to be placed and to represent the curve on the image, the desired curve corresponding to an interconnecting member for the at least three spinal implants 52. The at least two virtual implants are selected from a database of virtual implants to correspond to the at least three spinal implants 54. Once selected, the user is to place the virtual implant at a desired location in the image 56. This is done by aligning the at least two virtual implants with the desired curve while taking into account a position of a preceding virtual implant to place a subsequent virtual implant. Finally, the at least two spinal implants are placed according to the virtual implants in the image using the computer assisted surgery system 58. When a subsequent implant is positioned, the position of a preceding virtual implant is taken into account in order to place the subsequent virtual implant. Automated planning tools are used to determine the position or shape of the subsequent virtual implant with respect to the preceding virtual implant.

To illustrate the method, the case of a spinal intervention is used. If a first virtual implant is a pedicle screw, the surgeon selects it from the database and places it in the image. The second virtual implant can also be a pedicle screw. However its placement is determined based on the position and orientation of the first virtual pedicle screw. If a straight alignment is desired, then the second pedicle screw is placed so as to obtain a straight line from the first pedicle screw to the second pedicle screw. If a third virtual implant is a rod to be fitted on the screws, the shape of the rod is determined based on the placement of the first two pedicle screws. If the anatomy is limiting and doesn't allow many configurations or shapes for the rod, then the virtual rod is placed in the image according to the constraints of the anatomy and the virtual pedicle screws are then adjusted based on the position of the rod.

Therefore, lines are used to join together the virtual implants and align them on the image. The method also comprises calculating a location for the subsequent virtual implant based on a location of the preceding virtual implant and re-adjusting a position of a preceding virtual implant to better position the subsequent virtual implant. The last step of the method consists in placing the real implants based on the position of the virtual implants 58.

In an alternate embodiment, other interventions like intramedullary nailing may be addressed with these planning tools. In this case, the planning tools can be used to align the intramedullary rod with the proximal and distal nails during a fracture correction surgical intervention in order to allow the least invasive method without the presence of a cumbersome mechanical jig. Once the tracked rod is placed in the bone, virtual nails with graphic aiming devices can be placed to orient the positioning of the real implants such that they can pass through the holes in the rod (normally not visible to the surgeon). Additionally, in the case of a multi-fragment fracture, similar planning methods could be used to reposition virtual fragments obtained from intra-operative imaging and apply virtual nails or other relevant implants. The potential interventions cover all surgeries with multiple implants including but not restricted to orthopedics (spine, hip, knee, shoulder, etc) and ear-nose-throat (ENT).

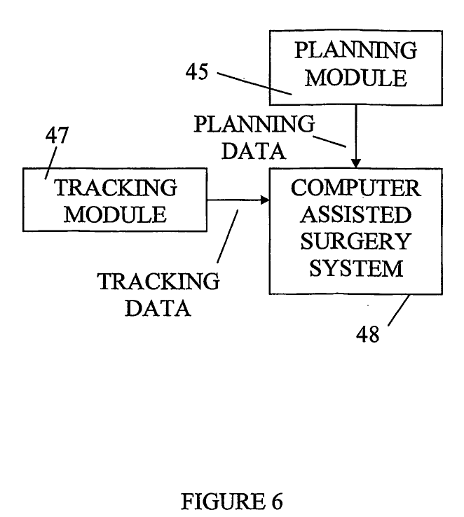

In a preferred embodiment, the planning module 45 is used with a computer assisted surgery system 48 and a tracking module 47, as illustrated in FIG. 6.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

1. An apparatus for planning a surgery, the apparatus comprising:
a display for an image representing a patient's anatomy;
a database of virtual implants from which a user selects;
a tool for said user to manipulate in order to select said virtual implants from said database and place said virtual implants in said image at desired locations; and
a positioning module adapted to calculating a position of a first of said virtual implants with respect to a second of said virtual implants and allow said user to align said first and second virtual implants with respect to each other, adapted to generating relative position data as a function of said calculated position, and adapted to sending said relative position data to said display.

2. An apparatus as claimed in claim 1, wherein said calculating a position comprises determining how well said virtual implants fit along a curve representing an interconnecting member for said virtual implants.

3. An apparatus as claimed in claim 1, wherein said surgery is a spinal surgery, said virtual implants are at least two spinal implants, and said positioning module is for aligning said at least two spinal implants along a curve representing an interconnecting member for said spinal implants.

4. An apparatus as claimed in claim 1, wherein said tool allows said user to input a desired relative position of said first virtual implant with respect to said second virtual implant, and said positioning module updates a position of at least one of said first virtual implant and said second virtual implant as a function of said desired relative position.

5. An apparatus as claimed in claim 1, wherein said tool allows said user to group together a plurality of virtual implants and input a desired relative position of said plurality of virtual implants with respect to another of said virtual implants, and said positioning module updates a position of at least one of said plurality of virtual implants and said another virtual implant as a function of said desired relative position.

6. An apparatus as claimed in claim 1, wherein said positioning module updates a position of a first virtual implant after said second virtual implant has been placed by said user at said desired location as a function of a predetermined relative position criteria.

7. An apparatus as claimed in claim 1, wherein said relative position data is graphically represented by said display.

8. An apparatus as claimed in claim 1, wherein said display is for displaying a fluoroscopic image representing said patient's anatomy.

9. An apparatus as claimed in claim 8, wherein said display updates said image every time a new fluoroscopic image is taken of said patient's anatomy.

10. An apparatus as claimed in claim 1, wherein said relative position data comprises an entry point of said virtual implants in said anatomy.

11. An apparatus as claimed in claim 1, wherein said relative position data comprises orientation of said virtual implants in said anatomy.

12. An apparatus as claimed in claim 1, wherein said relative position data comprises depth information of said virtual implants in said anatomy.

13. (canceled)

14. A method for placing at least two spinal implants during a surgery using a computer assisted surgery system, the method comprising:
providing an image representing a patient's anatomy;
determining a desired curve along which said at least two spinal implants are to be placed and representing said curve on said image, said desired curve corresponding to an interconnecting member for said at least two spinal implants;
selecting at least two virtual implants from a database of virtual implants to correspond to said at least two spinal implants;
placing said at least two virtual implants on said desired curve in said image by aligning said at least two virtual implants with said desired curve while taking into account a position of a preceding virtual implant to place a subsequent virtual implant; and
placing said at least two spinal implants according to said virtual implants in said image using said computer assisted surgery system.

15. A method as claimed in claim 14, wherein said placing said at least two virtual implants comprises using lines to join together said virtual implants and align them on said image representing a patient's anatomy.

16. A method as claimed in claim 14, wherein said placing said at least two virtual implants comprises calculating a location for said subsequent virtual implant based on a location of said preceding virtual implant.

17. A method as claimed in claim 14, wherein said selecting said at least two virtual implants comprises selecting said subsequent virtual implant having one of a position and a shape based on constraints imposed by said preceding virtual implant.

18. A method as claimed in claim 14, wherein said placing said at least two virtual implants comprises re-adjusting a position of said preceding virtual implant to better position said subsequent virtual implant in order to achieve an optimal alignment of all of said virtual implants.

19. A method as claimed in claim 14, wherein said at least two virtual implants are three virtual implants, and said interconnecting member is a rod to interconnect three spinal implants.

20. A method as claimed in claim 19, wherein said placing said at least two virtual implants comprises grouping together two of said three virtual implants and positioning said two virtual implants according to a desired relative position to at least one other virtual implant.

21. A method as claimed in claim 14, wherein said placing said at least two virtual implants comprises determining at least one of an entry point, a depth, and an orientation of each of said virtual implants on said anatomy.

22. A method as claimed in claim 14, wherein said placing said at least two virtual implants comprises placing according to predetermined relative position criteria.

23. A method as claimed in claim 14, wherein said providing an image comprises providing a fluoroscopic image.

24. A method as claimed in claim 23, wherein said placing said at least two spinal implants comprises updating said fluoroscopic image after each of said at least two spinal implants has been placed.

25. (canceled)

26. (canceled)
